(19) United States
(12) Patent Application Publication
(10) Pub. No.: US 2007/0073361 A1
(43) Pub. Date: Mar. 29, 2007

(54) Medical device for restoration of autonomic and immune functions impaired by neuropathy

(75) Inventors: Andy Ofer Goren, Newport Beach, CA (US); Yehuda G. Goren, Scotts Valley, CA (US); Peter Novak, Jamaica Plain, MA (US); Elliott J. Stein, Morristown, NJ (US); Christopher Chi-Chuen Chen, Wallace, CA (US); Amy Morningstar, Scotts Valley, CA (US)

Correspondence Address: THELEN REID & PRIEST, LLP, P. O. BOX 640640, SAN JOSE, CA, 95164-0640, US

(73) Assignee: BioQ, Inc., Newport Beach, CA (US)

(21) Appl. No.: 11/526,206

(22) Filed: Sep. 22, 2006

Related U.S. Application Data
(60) Provisional application No. 60/719,812, filed on Sep. 23, 2005.

(51) Int. Cl. A61N 1/00; A61B 5/02; A61B 5/04

(52) U.S. Cl. 607/62; 600/509; 600/544; 600/500

(57) ABSTRACT

A device and method for treatment of impairments relating to neuropathy rely on sensory substitution to train a patient to associate an affected condition with stimuli that are generated based on detection of the condition.

### CROSS-REFERENCE TO THE APPLICATIONS

This application claims the benefit of U.S. provisional patent application no. 60/719,812, filed on Sep. 23, 2005, incorporated herein by reference.

### BACKGROUND OF THE INVENTION

1. Field of the Invention

The invention relates to the treatment of autonomic and immune disorders due to neuropathy.

2. Description of Related Art

Autonomic impairment and abnormalities of the immune system are common. They are frequently seen in geriatric patients and are often associated with prevalent disorders such as diabetes. Autonomic and immune system impairment frequently results in severe disability. For example, autonomic neuropathy in diabetes decreases patients survival and it is estimated that 25%-50% of patients with symptomatic autonomic impairment die within 5 to 10 years of diagnosis.

The leading cause of death in diabetic patients with autonomic disorders is heart disease and abnormalities in vascular system. Cardiovascular autonomic neuropathy, which affects approximately 20% of diabetic patients, is a leading cause of cardiac arrhythmias, postural hypotension, asymptomatic ischemia, and exercise intolerance. During daily activities the autonomic nervous system controls heart rate and vascular dynamics. The autonomic nervous system receives afferent information from the heart as well as various receptors distributed throughout the human body such as the baroreceptors in the aortic arch and carotid arteries. Integrating the afferent input information, the autonomic nervous system controls heart rate and vascular dynamics via efferent fibers. In patients with cardiovascular autonomic neuropathy, afferents, efferents, or both systems may function improperly. In some cases such as orthostatic hypotension, which also affects non-diabetic elderly patients as well as patients suffering from atherosclerosis, the baroreceptors malfunction and/or loss of autonomic-mediated postural adjustment of the vascular resistance lead to increased incidence of falls, loss of consciousness, dizziness and a myriad of debilitating conditions.

Neuropathies affecting the autonomic and sensory fibers lead to a wide array of disorders such as orthostatic hypotension, arrhythmias, silent myocardial infract, respiratory dysfunction, esophageal dysfunction, neuropathic bladder (voiding dysfunction due to sensory and/or autonomic neuropathy), erectile dysfunction, and tachycardia. The variety of conditions attributed to autonomic impairments reflects the variety of body functions controlled by the autonomic nervous system. For example, esophageal dysfunction due to neuropathy is often the result of diminished sensation in the esophagus leading to abnormal or difficulty in swallowing. Another example is neuropathic bladder where voiding dysfunction is due to sensory and autonomic neuropathy and results in for example diminished bladder sensation, and/or decreased bladed contractility. The spectrum of voiding symptoms include dribbling, alterations of urinary frequency, incontinence, and urinary infections.

The immune system is also regulated by the central nervous system. Conditions such as inflammation in patients with arthritis can be reduced by proper controlling of signal molecules, such as TNF reduction, by the nervous system. However, chronic inflammation often leads to neuropathy and thus impaired nervous system regulation of the immune system leading to further deterioration of immune system functions.

Various treatments are available for different autonomic symptoms. Most treatments for autonomic impairment are pharmacological. Due to the complexity of the nervous system and the inherent properties of drugs, pharmacological treatments are frequently accompanied by severe side effects. For example, orthostatic hypotension is treated by fludrocortisone and proamatine. These drugs are effective; however, their use can provoke end-organ damage including congestive heart failure and renal failure.

There therefore exists a need for a system that addresses the limitations of previous approaches by providing a wearable, low cost, non-invasive device that stimulates a patient's perception modality so as to provide the nervous system with stimulus indicative of the information not received by the nervous system due to neuropathy.

### SUMMARY OF THE INVENTION

The current invention overcomes the limitations of previous treatments by providing a wearable, low cost, non-invasive device that stimulates a patient's perception modality so as to provide the central nervous system with stimulus indicative of the information not received by the nervous system due to neuropathy.

The current invention makes use of the phenomena of sensory substitution. Sensory substitution is a well known neurological phenomenon whereby a subject with a failed or degraded mode of perception learns that an input signal from a different modality of perception on the subject's body is used to complement the failed or degraded perception.

In accordance with one embodiment of the invention, there is provided a device for treating neuropathic bladder due to sensory neuropathy. The device includes one or more sensors configured to generate signals in response to the amount of fluid in a human bladder, a controller configured to determine the timing for bladder emptying using the amount of fluid in the bladder signals and to issue control signals at the proper timing for bladder emptying, and one or more stimulators configured to stimulate a wearer of the device in response to the control signal.

In accordance with another embodiment of the invention, there is provided a device for treating diabetic esophageal dysfunction due to sensory neuropathy. The device includes one or more sensors configured to generate signals in response to the location of food in a human esophagus, a controller configured to determine the location of the food in the esophagus using the location signals and to issue control signals in accordance with the food location, and one or more stimulators configured to stimulate a wearer of the device in response to the control signal.

In accordance with yet another embodiment of the invention, there is provided a device for treating silent myocardial infarct due to sensory neuropathy. The device includes one or more sensors configured to generate signals in response to cardiac events, a controller configured to determine abnormal cardiac events using the cardiac events signals and to issue control signals at the onset of an abnormal cardiac event, and one or more stimulators configured to stimulate a wearer of the device in response to the control signal.

The preferred embodiment of the current invention is a non-invasive device; however, the current invention could be implanted and used to directly stimulate afferent and efferent nerves.

### BRIEF DESCRIPTION OF THE SEVERAL VIEWS OF THE DRAWINGS

Many advantages of the present invention will be apparent to those skilled in the art with a reading of this specification in conjunction with the attached drawings, wherein like reference numerals are applied to like elements, and wherein:

FIG. 1 is a schematic diagram illustrating an embodiment of the invention;

FIG. 2 illustrates an necklace-type embodiment of the invention;

FIG. 3 illustrates and embodiment having a necklace and a behind-the-ear component; and

FIG. 4 illustrates a general method in accordance with an embodiment of the invention.

### DETAILED DESCRIPTION OF THE INVENTION

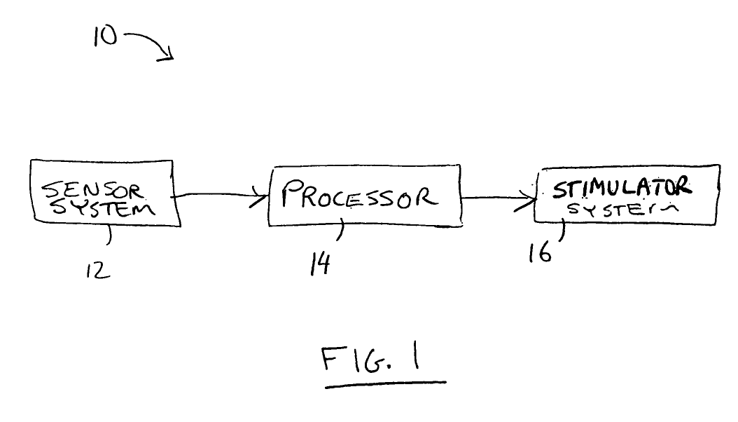

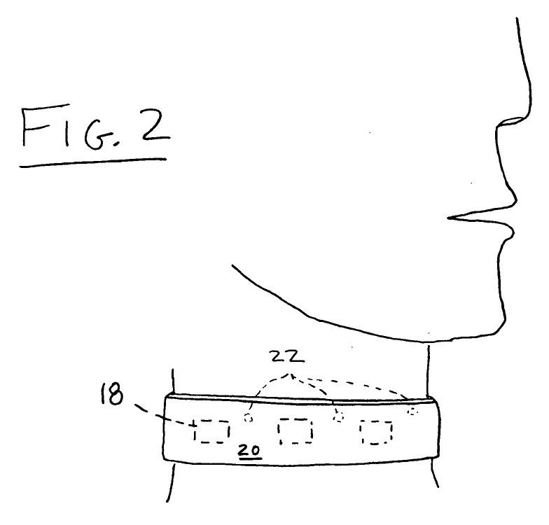

FIG. 1 is a schematic diagram of a therapeutic system 10 in which a sensor system 12 provides information to a processor 14 which is used to activate a stimulator system 16. The sensor system 12 consists of one or more sensors adapted to provide information representative of various physiological conditions, depending on the specific application. For instance, in the treatment of orthostatic hypotension, the sensors can take the form of inclinometers which extract information relating to the head position of a wearer of a collar, necklace, or chest patch in which they are placed. This is illustrated in FIG. 2, in which inclinometers 18 are provided in a necklace 20 worn around the neck of a patient. The information extracted by the sensor system 12 is used to supplement information from compromised baroreceptors of the wearer caused by neuropathy or other conditions. Alternatively or in addition, blood pressure measurements from blood pressure detectors (not shown) operating in conjunction with processor 14 can be conducted to provide blood pressure information. Other types of sensors that can be part of sensor system 12 and operate in conjunction with processor 14, or have their own processor or logic, are gyroscopes, accelerometers, pressure sensors, pulse detectors, piezoelectric sensors, oximeters to measure blood oxygen, sweat/moisture detectors, light/optical detectors, acoustic sensors, sonar sensors, electrocardiogram sensors, electroencephalogram sensors, sensors of blood glucose or other chemicals or molecules, sensors configured to detect human breathing parameters, conditions relating hypoglycemia, progress of swallowing through the esophagus, progress of digestion in the stomach and GI track, and so forth. The sensors can be placed at various positions on the patient and are not limited to the neck, and can be used to detect movement of body parts of the patient, including head motion, limb vibration, and so forth. They can also detect the posture of the patient.

Processor 14 uses signals from sensor system 12 to control stimulation system 16. Stimulation system 16 includes for example vibratory stimulators 22 that provide mechanical supra-threshold neuronal stimulation to skin mechanoreceptors. Such stimulation can for example be vibration. Stimulators 22 can also of a type that provides transcutaneous electrical stimulation to the skin mechanoreceptors. They can also provide electrical stimulation to at least one efferent nerve, in which case they can be implantable in the body of the patient proximal to the particular efferent nerve. They can also provide mechanical pressure to a body part of the patient, or provide auditory/hearing aid, visual, vibratory mechanical, olfactory, taste, heat/cold, or pain stimulation. Alternatively or in addition, the stimulators 22 can be separated from the other components and can communicate therewith wirelessly or via a wired link..

While shown to be part of a necklace 20, the sensors of sensor system 12 and the stimulators 22 can be provided separately from the necklace in contact with other parts of the patient's body. Communication between the sensors and the controller 14 can take place wirelessly or using a wired link between the sensors and/or stimulators and the necklace or other wearable component in which the controller 14 resides. The device does not have to be in the form of a necklace, but can instead be a bracelet, anklet, patch, ring, earring, part of a hearing aid, implantable device, ornamental article such as jewelry, and so forth, and, as stated above, can be in the form of multiple components worn on different parts of the body and in communication with one another.

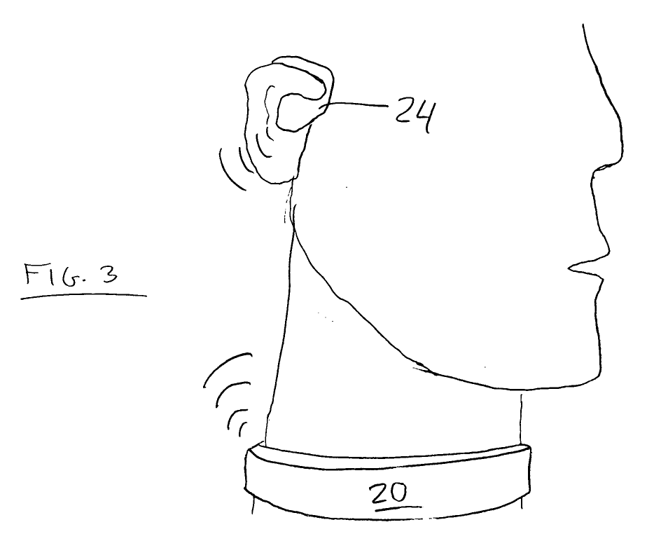

This is illustrated in FIG. 3, in which necklace 20 and a behind-the-ear device 24, in which the sensors, controller and stimulators are variously distributed depending on the patient characteristics to be measured and the type of stimulation to be applied, communicate wirelessly with one another in order to apply appropriate treatment for a particular autonomic impairment or immune disorder due to sensory neuropathy.

It is also contemplated that communication between the system 10 and a remote device, for instance a computer terminal operated by a physician or caretaker, can take place. In this manner operation and control of the system 10, along with monitoring of the patient, can be effected remotely from the remote terminal. Such communication can take place wirelessly or with a wired link, and can be by way of the Internet or a cellular or satellite network.

The system 10 includes a power source (not shown) for powering its various components. The power source can be electromechanical, or a battery pack that is rechargeable via an adapter or by connection to a computer or other device, for example by way of a USB or FireWire connection, or wirelessly by way of an induction coupling.

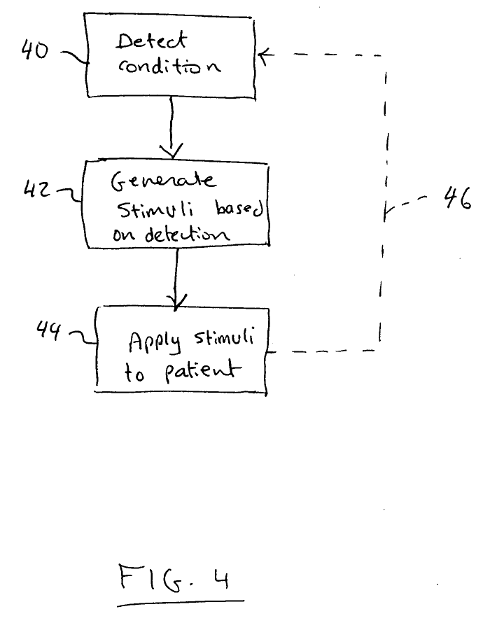

With reference to FIG. 4, it can be seen that in operation, the sensors from sensor system 12 are configured to detect a particular characteristic of the patient, in Step 40, and to provide a signal indicative of said characteristic. An example characteristic used for the treatment of orthostatic hypotension due to sensory neuropathy is body position change, which can be detected using tilt sensors or inclinometers (a type of accelerometers). A signal (or signals) indicative of the body position change is forwarded to the processor 14 from the sensor system 12. The processor 14 then uses the body position change signal to generate a stimulation signal (Step 42) commensurate in scope, degree, intensity, frequency, or any other feature, with the sensed body position change. The stimulation signal is applied to the stimulator system 16, and causes the stimulator system, and more particularly, one or more stimulators thereof, to issue stimuli to the patient that are commensurate with the body position change (Step 44). For instance, when the body is in a supine position, a first sensor signal is sent to the processor 14 from the sensor system 12. Processor 14 then issues a first stimulation signal causing a stimulator such as a vibrator 22 to generate vibrations of a first frequency. When the body position changes to an upright position, as when the patient changes from a supine position to a standing position, a second sensor signal is generated by the sensor system 12 and sent to processor 14. Processor 14 then issues a second stimulation signal to the vibrator 22, causing the vibrator to generate vibrations of a second frequency. Over time, and, likely, repeated iterations (dashed arrow 46 in FIG. 4), the patient's body “learns” to associate the first vibration frequency with a supine position, and the second vibration frequency with a change in position to an upright position, and becomes conditioned to respond in a physiologically appropriate manner—for example by increasing blood pressure, constricting peripheral vasculature, and so forth—in order to cope with the changing demands. Normally, these conditions would automatically be performed by the healthy human body, which would be aware of the body position change and which would adjust physiologically to changes in order to maintain proper body function such as blood supply and so forth. In patients that have impaired afferent input capability due to sensory neuropathy for instance, the central nervous system is not receiving accurate information regarding the change of position of the body, and is therefore unable to make the proper response. The system 10 ameliorates this lack of accurate information and provides information that the body learns to associate with characteristics it would normally detect and to properly respond. The arrangement of the stimulators 22 can be such that they are spatially separated in a manner that optimizes providing the patient, and specifically, the nervous system of the patient, with spatial information missing due to sensory neuropathy. Temporal separation can also be provided and controlled, by controller 14, so as to provide the nervous system with missing temporal and/or frequency information. Stimulation from stimulators 22 can be applied in a frequency-varying manner in order to provide the nervous system with the missing temporal and/or frequency. Variations in stimulation intensity duration, and so forth, can be applied for similar effect. A general method in accordance with an embodiment of the invention is illustrated in FIG. 4. In step 40, a condition of the patient is detected.

In the treatment of impaired bladder sensation in diabetic cystopathy due to sensory neuropathy, the sensors of system 12 are configured to detect the amount of fluid in the bladder of a patient which can be detected using fluid ultrasound sensors. A signal (or signals) indicative of the amount of fluid in a patient bladder is forwarded to the processor 14 from the sensor system 12. The processor 14 then uses the amount of fluid signal to generate a stimulation signal commensurate in scope, degree, intensity, frequency, or any other feature, with the amount of fluid in the bladder of a patient. The stimulation signal is applied to the stimulator system 16, and causes the stimulator system, and more particularly, one or more stimulators thereof, to issue stimuli to the patient that are commensurate with the amount of fluid in the bladder of the patient. For instance, when the bladder is less then 5% full, a first sensor signal is sent to the processor 14 from the sensor system 12. Processor 14 then issues a first stimulation signal causing a stimulator such as a vibrator 22 to generate vibrations of a first frequency. When the bladder is more then 90% full, a second sensor signal is generated by the sensor system 12 and sent to processor 14. Processor 14 then issues a second stimulation signal to the vibrator 22, causing the vibrator to generate vibrations of a second frequency. Over time, the patient's body “learns” to associate the first vibration frequency with an almost empty bladder, and the second vibration frequency with an almost full bladder, and becomes conditioned to respond in a physiologically appropriate manner—for example by urinating or ceasing to drink additional fluids, and so forth—in order to cope with the changing demands. Normally, these conditions would automatically be performed by the healthy human body, which would be aware of the amount of fluid in the bladder and which would adjust physiologically to changes in order to maintain proper body function. In patients that have impaired afferent input capability due to sensory neuropathy for instance, the central nervous system is not receiving accurate information regarding the amount of fluid in the bladder, and is therefore unable to make the proper response. The system 10 ameliorates this lack of accurate information and provides information that the body learns to associate with characteristics it would normally detect and to properly respond.

In the treatment of diabetic esophageal dysfunction due to sensory neuropathy, the sensors of system 12 are configured to detect the location of food in the esophagus of a patient which can be detected using ultrasound sensors or pressure sensors. A signal (or signals) indicative of the location of food in the esophagus of a patient is forwarded to the processor 14 from the sensor system 12. The processor 14 then uses the location of food signal to generate a stimulation signal commensurate in scope, degree, intensity, frequency, or any other feature, with the location of food in the esophagus of a patient. The stimulation signal is applied to the stimulator system 16, and causes the stimulator system, and more particularly, one or more stimulators thereof, to issue stimuli to the patient that are commensurate with location of food in the esophagus of a patient. For instance, when the food is at the top portion of the esophagus, a first sensor signal is sent to the processor 14 from the sensor system 12. Processor 14 then issues a first stimulation signal causing a stimulator such as a vibrator 22 to generate vibrations of a first frequency. When the food is at half the length of the esophagus, a second sensor signal is generated by the sensor system 12 and sent to processor 14. Processor 14 then issues a second stimulation signal to the vibrator 22, causing the vibrator to generate vibrations of a second frequency. Over time, the patient's body “learns” to associate the first vibration frequency with food at the top of the esophagus, and the second vibration frequency with food at half the length of the esophagus, and becomes conditioned to respond in a physiologically appropriate manner—for example by contracting the esophageal muscles more quickly, and so forth—in order to cope with the changing demands. Normally, these conditions would automatically be performed by the healthy human body, which would be aware of the location of food in the esophagus and which would adjust physiologically to changes in order to maintain proper body function. In patients that have impaired afferent input capability due to sensory neuropathy for instance, the central nervous system is not receiving accurate information regarding the location of food in the esophagus, and is therefore unable to make the proper response. The system 10 ameliorates this lack of accurate information and provides information that the body learns to associate with characteristics it would normally detect and to properly respond.

In the treatment of arrhythmias due to sensory neuropathy, the sensors of system 12 are configured to detect the heart rhythm of a patient which can be detected using a electrocardiogram sensors or pressure sensors. A signal (or signals) indicative of the heart rhythm of a patient is forwarded to the processor 14 from the sensor system 12. The processor 14 then uses the heart rhythm signal to generate a stimulation signal commensurate in scope, degree, intensity, frequency, or any other feature, with the heart rhythm of a patient. The stimulation signal is applied to the stimulator system 16, and causes the stimulator system, and more particularly, one or more stimulators thereof, to issue stimuli to the patient that are commensurate with the heart rhythm of a patient. For instance, when the heart rhythm becomes abnormal, a first sensor signal is sent to the processor 14 from the sensor system 12. Processor 14 then issues a first stimulation signal causing a stimulator such as a vibrator 22 to generate vibrations of a first frequency. When the hearth rhythm returns to normal, a second sensor signal is generated by the sensor system 12 and sent to processor 14. Processor 14 then issues a second stimulation signal to the vibrator 22, causing the vibrator to generate vibrations of a second frequency. Over time, the patient's body “learns” to associate the first vibration frequency with the onset of an abnormal heart rhythm, and the second vibration frequency with the return of normal heart rhythm, and becomes conditioned to respond in a physiologically appropriate manner—for example by influencing the heart rate, and so forth—in order to cope with the changing demands. Normally, these conditions would automatically be performed by the healthy human body, which would be aware of the heart rhythm and which would adjust physiologically to changes in order to maintain proper body function. In patients that have impaired afferent input capability due to sensory neuropathy for instance, the central nervous system is not receiving accurate information regarding the heart rhythm, and is therefore unable to make the proper response. The system 10 ameliorates this lack of accurate information and provides information that the body learns to associate with characteristics it would normally detect and to properly respond.

In the treatment of silent myocardial infarct due to sensory neuropathy, the sensors of system 12 are configured to detect various ECG parameters such as ST segment and Q waves which can be detected using a electrocardiogram (ECG) sensors. A signal (or signals) indicative of the ECG parameters of a patient is forwarded to the processor 14 from the sensor system 12. The processor 14 then uses the ECG parameters signal to calculate the likelihood of a patient suffering from a myocardial infarct and generate a stimulation signal commensurate in scope, degree, intensity, frequency, or any other feature, with the likelihood of a patient suffering from a myocardial infarct. The stimulation signal is applied to the stimulator system 16, and causes the stimulator system, and more particularly, one or more stimulators thereof, to issue stimuli to the patient that are commensurate with the likelihood of a patient suffering from a myocardial infarct. For instance, when the processor 14 detects that the ST-segment elevation is greater than 1 mm in 2 anatomically contiguous leads or new Q waves signal are detected from the sensor system 12. Processor 14 then issues a stimulation signal causing a stimulator such as a vibrator 22 to generate mechanical vibrations of a first fixed frequency. If the processor 14 detects a T-wave inversion, an ST-segment depression, or an abnormal ST-T wave signal from the sensor system 12,processor 14 issues a stimulation signal causing a stimulator such as a vibrator 22 to generate mechanical vibrations of a second fixed frequency. The patient “learns” to associate the first vibration frequency with a high likelihood of an onset of a myocardial infarct and the second frequency with an intermediate likelihood of an onset of a myocardial infract and is able to respond in an appropriate manner—for example by seeking help or taking medications, and so forth—in order to cope with the condition. Normally, these conditions would automatically trigger a pain response by the human body, which would be aware of the myocardial infarct and which would adjust physiologically to changes in order to maintain proper body function. In patients that have impaired afferent input capability due to sensory neuropathy for instance, the central nervous system is not receiving accurate information regarding the pain from a myocardial infarct, and is therefore unable to make the proper response. The system 10 ameliorates this lack of accurate information and provides information that the body learns to associate with characteristics it would normally detect and to properly respond.

In the treatment of abnormal pulmonary reflexes and respiratory problems due to neuropathy of afferent fibers, the sensors of system 12 are configured to detect the lung volume of a patient or blood oxygen level which can be detected using spirometer sensors or oximeter sensors, respectively. A signal (or signals) indicative of the amount of oxygen in the blood of a patient is forwarded to the processor 14 from the sensor system 12. The processor 14 then uses the blood oxygen signal to generate a stimulation signal commensurate in scope, degree, intensity, frequency, or any other feature, with the amount of oxygen in the blood of a patient. The stimulation signal is applied to the stimulator system 16, and causes the stimulator system, and more particularly, one or more stimulators thereof, to issue stimuli to the patient that are commensurate with the amount of oxygen in the blood of a patient. For instance, when the oxygen level becomes low, a first sensor signal is sent to the processor 14 from the sensor system 12. Processor 14 then issues a first stimulation signal causing a stimulator such as a vibrator 22 to generate vibrations of a first frequency. When the oxygen level returns to normal, a second sensor signal is generated by the sensor system 12 and sent to processor 14. Processor 14 then issues a second stimulation signal to the vibrator 22, causing the vibrator to generate vibrations of a second frequency. Over time, the patient's body “learns” to associate the first vibration frequency with the low blood oxygen level, and the second vibration frequency with the return of normal blood oxygen level, and becomes conditioned to respond in a physiologically appropriate manner—for example by influencing the breathing pattern, and so forth—in order to cope with the changing demands. Normally, these conditions would automatically be performed by the healthy human body, which would be aware of the lung pressure as well as blood oxygen and which would adjust physiologically to changes in order to maintain proper body function. In patients that have impaired afferent input capability due to sensory neuropathy for instance, the central nervous system is not receiving accurate information regarding the lung pressure, and is therefore unable to make the proper response. The system 10 ameliorates this lack of accurate information and provides information that the body learns to associate with characteristics it would normally detect and to properly respond.

In the treatment of immune disorders due to sensory neuropathy such as arthritis, the sensors of system 12 is configured to detect a chemical or biological compound in a patient which can be detected using spectrometer sensors. A signal (or signals) indicative of the amount of the compound detected in a sample from a patient is forwarded to the processor 14 from the sensor system 12. The processor 14 then uses the amount of the compound detected signal to generate a stimulation signal commensurate in scope, degree, intensity, frequency, or any other feature, with the amount of the compound detected in the sample from a patient. The stimulation signal is applied to the stimulator system 16, and causes the stimulator system, and more particularly, one or more stimulators thereof, to issue stimuli to the patient that are commensurate with the amount of the compound detected in the sample from a patient. For instance, when the amount of p38 MAP kinase becomes high, a first sensor signal is sent to the processor 14 from the sensor system 12. Processor 14 then issues a first stimulation signal causing a stimulator such as a vibrator 22 to generate vibrations of a first frequency. When the amount of p38 MAP kinase returns to normal, a second sensor signal is generated by the sensor system 12 and sent to processor 14. Processor 14 then issues a second stimulation signal to the vibrator 22, causing the vibrator to generate vibrations of a second frequency. Over time, the patient's body “learns” to associate the first vibration frequency with the onset of an abnormal inflammation response, and the second vibration frequency with the return of the body to the normal state, and becomes conditioned to respond in a physiologically appropriate manner—for example by influencing the production of TNF, and so forth—in order to cope with the changing demands. Normally, these conditions would automatically be performed by the healthy human body, which would be aware of the over reacting immune response and which would adjust physiologically to changes in order to maintain proper body function. In patients that have impaired afferent input capability due to sensory neuropathy for instance, the central nervous system is not receiving accurate information regarding the over reactive immune system, and is therefore unable to make the proper response. The system 10 ameliorates this lack of accurate information and provides information that the body learns to associate with characteristics it would normally detect and to properly respond.

The above are exemplary modes of carrying out the invention and are not intended to be limiting. It will be apparent to those of ordinary skill in the art that modifications thereto can be made without departure from the spirit and scope of the invention as set forth in the following claims.

1. A device for restoring in a human patient autonomic nervous system functions impaired by neuropathy comprising:
one or more sensors configured to generate sensor signals in response to a characteristic associated with the patient;
a controller configured to receive the sensor signals and to issue stimulation signals based on the sensor signals; and
one or more stimulators configured to stimulate the patient in response to said stimulation signals.

2. The device of claim 1 wherein at least one sensor is an accelerometer and/or inclinometer.

3. The device of claim 1 wherein at least one sensor is a gyroscope.

4. The device of claim 1 wherein at least one sensor is a pressure sensor.

5. The device of claim 1 wherein at least one sensor is a piezoelectric sensor.

6. The device of claim 1 wherein at least one sensor is configured to detect the motion of a human body part.

7. The device of claim 1 wherein at least one sensor is configured to detect the motion of a human head.

8. The device of claim 1 wherein at least one sensor is configured to detect the change in posture of a human patient.

9. The device of claim 1 wherein at least one sensor is an oximeter.

10. The device of claim 1 wherein at least one sensor is configured to detect human sweat.

11. The device of claim 1 wherein at least one sensor is a light and/or optical sensor.

12. The device of claim 1 wherein at least one sensor is an acoustic sensor.

13. The device of claim 1 wherein at least one sensor is a sonar sensor.

14. The device of claim 1 wherein at least one sensor is a electrocardiogram sensor.

15. The device of claim 1 wherein at least one sensor is a electroencephalogram sensor.

16. The device of claim 1 wherein at least one sensor is a pulse detector.

17. The device of claim 1 wherein at least one sensor is configured to monitor human breathing parameters.

18. The device of claim 1 wherein at least one sensor is configured to monitor human blood glucose level

19. The device of claim 1 wherein at least one sensor is configured to monitor conditions related to Hypoglycemia.

20. The device of claim 1 wherein at least one sensor is configured to monitor shaking of at least one human limb.

21. The device of claim 1 wherein at least one sensor is configured to monitor the progress of swallowing through the human esophagus.

22. The device of claim 1 wherein at least one sensor is configured to monitor the progress of digestion in the human stomach.

23. The device of claim 1 wherein at least one sensor is configured to monitor the temperature of a localized region of the human body and/or the temperature of the entire human body.

24. The device of claim 1 wherein at least one sensor is a spectrometer.

25. The device of claim 1 wherein at least one stimulator is configured to provide mechanical supra threshold neuronal stimulation to the skin mechanoreceptors.

26. The device of claim 1 wherein at least one stimulator is configured to provide transcutaneous electrical stimulation to the skin mechanoreceptors.

27. The device of claim 1 wherein at least one stimulator is configured to provide electrical stimulation to at least one afferent nerve.

28. The device of claim 1 wherein at least one stimulator is configured to provide mechanical pressure to a human body part.

29. The device of claim 1 wherein at least one stimulator is configured to provide auditory stimulation.

30. The device of claim 1 wherein at least one stimulator is configured to provide visual stimulation.

31. The device of claim 1 wherein at least one stimulator is configured to provide vibratory mechanical stimulation.

32. The device of claim 1 wherein at least one stimulator is configured to provide olfactory stimulation.

33. The device of claim 1 wherein at least one stimulator is configured to provide taste stimulation.

34. The device of claim 1 wherein at least one stimulator is configured to provide heat/cold stimulation.

35. The device of claim 1 wherein at least one stimulator is configured to provide pain stimulation to a human body part.

36. The device of claim 1 wherein the device is a patch worn on the human body.

37. The device of claim 1 wherein at least a sensor, controller or stimulator is disposed in a necklace.

38. The device of claim 1 wherein at least a sensor, controller or stimulator is disposed in a bracelet.

39. The device of claim 1 wherein at least a sensor, controller or stimulator is disposed in a ring.

40. The device of claim 1 wherein at least a sensor, controller or stimulator is disposed in an anklet.

41. The device of claim 1 wherein at least a sensor, controller or stimulator is disposed in an earring.

42. The device of claim 1 wherein the device is worn on the human body.

43. The device of claim 1 wherein the device is in the shape an ornamental jewelry article.

44. The device of claim 1 wherein the device is embedded in a hearing aid.

45. The device of claim 1, further comprising:
a first wearable component in which is disposed at least one sensor; and
a second wearable component in which is disposed at least one stimulator,
wherein the controller is disposed in one of the first or second wearable components and communicates wirelessly or via wired means with at least one sensor and/or at least one stimulator.

46. The device of claim 1 wherein the controller is programmable.

47. The device of claim 1 wherein the controller uses the information provided by the one or more sensors to predict the timing and/or phase of a missing stimulus due to neuropathy.

48. The device of claim 1 wherein the controller employs algorithms for adaptive learning.

49. The device of claim 1 wherein the controller is programmable via a computer connection.

50. The device of claim 1 wherein the controller includes a wireless transmitter and receiver.

51. The device of claim 50 wherein the controller communicates with an external computer to provide information for a physician.

52. The device of claim 50 wherein the controller communicates with one or more devices located at different locations on the human body.

53. The device of claim 52 wherein the communication with the one or more devices is used to synchronize at least one stimulator with at least one sensor.

54. The device of claim 1 wherein at least one stimulator has adjustable stimulation strength.

55. The device of claim 1 wherein one or more stimulators are arranged spatially such that the nervous system of the patient is provided with spatial information missing due to neuropathy.

56. The device of claim 1 wherein one or more stimulators provide time varying stimulation such that the nervous system of the patient is provided with temporal and/or frequency information missing due to neuropathy.

57. The device of claim 1 wherein one or more stimulators provide frequency varying stimulation such that the nervous system of the patient is provided with temporal and/or frequency information missing due to neuropathy.

58. The device of claim 1 used for the treatment of orthostatic hypotension.

59. The device of claim 1 used for the treatment of cardiac arrhythmias and/or cardiac disorders due to neuropathy.

60. The device of claim 1 used for the treatment of Cystopathy.

61. The device of claim 1 used for the treatment of breathing disorders.

62. The device of claim 1 used for the treatment of Hypoglycemia.

63. The device of claim 1 used for the treatment of digestive disorders.

64. The device of claim 1 used for the treatment of swallowing disorders.

65. The device of claim 1 used for the treatment of urinary disorders due to neuropathy.

66. The device of claim 1 used for the treatment of sweat disorders.

67. The device of claim 1 used for the treatment of body temperature regulation disorders.

68. The device of claim 1 used for the treatment of pupil disorders.

69. The device of claim 1 used for the treatment of autonomic disorders arising from neuropathy due to aging.

70. The device of claim 1 used for the treatment of autonomic disorders arising from neuropathy due to chemotherapy.

71. The device of claim 1 used for the treatment of autonomic disorders arising from neuropathy due to HIV/AIDS.

72. The device of claim 1 used for the treatment of disorders arising from Parkinson's disease.

73. The device of claim 1 used for the treatment of immune disorders.

74. The device of claim 1 used for the treatment of inflammation disorders.

75. A method for treating autonomic neuropathy in a patient comprising:
for a first body function that influences control of a second body function by the central nervous system, detecting a characteristic of the first body function;
generating one or more stimuli in accordance with said detected characteristic; and
applying said one or more stimuli to the patient such that the patient is provided with an association of said stimuli to said first characteristic and with control of the second body function by the central nervous system.

76. A method for treating silent myocardial infarct relating to sensory neuropathy, comprising:
detecting ECG (electrocardiogram) parameters of a patient;
determining from said detecting the likely hood of a myocardial infarct;
generating one or more stimuli in accordance with said determining;
applying said one or more stimuli to the patient; and
causing the patient to associate said one or more stimuli with the likelihood of myocardial infract through sensory substitution.

77. A method for treating conditions arising from impaired bladder sensation, comprising:
detecting an amount of fluid in the bladder of a patient;
generating one or more stimuli in accordance with said detecting;
applying said one or more stimuli to the patient; and
causing the patient to associate said one or more stimuli with the amount of fluid in the bladder through sensory substitution.

78. A method for treating diabetic esophageal dysfunction relating to sensory neuropathy, comprising:
detecting the location of food in the esophagus of a patient;
generating one or more stimuli in accordance with said detecting;
applying said one or more stimuli to the patient; and
causing the patient to associate said one or more stimuli with the location of food in the esophagus through sensory substitution.

79. A method for treating arrhythmias relating to sensory neuropathy, comprising:
detecting the heart rhythm of a patient;
generating one or more stimuli in accordance with said detecting;
applying said one or more stimuli to the patient; and
causing the patient to associate said one or more stimuli with the heart rhythm through sensory substitution.

80. A method for treating abnormal pulmonary reflexes and/or respiratory problems relating to neuropathy of afferent fibers, comprising:
detecting at least one of lung volume or blood oxygen of a patient;
generating one or more stimuli in accordance with said detecting;
applying said one or more stimuli to the patient; and
causing the patient to associate said one or more stimuli with the lung volume and/or blood oxygen through sensory substitution.

81. A method for treating immune impairment relating to sensor neuropathy, comprising:
detecting a chemical and/or biological compound associated with the immune response of a patient;
generating one or more stimuli in accordance with said detecting;
applying said one or more stimuli to the patient; and
causing the patient to associate said one or more stimuli with the immune response through sensory substitution.

82. The device of claim 1 wherein the device is implantable in a human body.

83. A method for treating baroreflex failure and/or orthostatic hypotension due to afferent sensory neuropathy or baroreceptor malfunction, the method comprising:
detecting the position and/or change of position of at least a portion of the body of a patient;
generating one or more stimuli in accordance with said detecting;
applying said one or more stimuli to the patient; and
causing the patient to associate said one or more stimuli with the position and/or change of position of said portion of the body through sensory substitution.

84. The method of claim 83 wherein position is a function of at least one of posture or orientation.
